(19) United States
(12) Patent Application Publication
(10) Pub. No.: US 2015/0335316 A1
(43) Pub. Date: Nov. 26, 2015

(54) MRI SYSTEM FOR ROBOTICALLY ASSISTED BREAST BIOPSY

(71) Applicant: General Electric Company, Schenectady, NY (US)

(72) Inventors: Robert David Darrow, Scotia, NY (US); Ileana Hancu, Clifton Park, NY (US)

(73) Assignee: General Electric Company, Schenectady, NY (US)

(21) Appl. No.: 14/285,836

(22) Filed: May 23, 2014

(51) Int. Cl. A61B 10/02; A61B 19/00

(52) U.S. Cl. CPC A61B 10/0233; A61B 19/2203; A61B 2010/0208; A61B 2019/2207

(57) ABSTRACT

A breast biopsy system utilizing a needle biopsy device configured for guidance by a robotic guidance device into a treatment position wherein the needle tip is positioned adjacent target tissue in patient. The system including an MRI compatible device localization system adapted to track one or more points on the needle biopsy device and generate real-time device localization data. A Magnetic Resonance Imaging (MRI) system provides a multi-planar reference image data from the patient being treated. The MRI system is connected to the MRI compatible device localization system and operable to display an overlay image, reconstructed from the real-time device localization data, on the multi-planar reference image data which depicts the location of the needle biopsy device relative to the target tissue in the patient. A method of performing a breast biopsy utilizing the disclosed breast biopsy system is also provided

### BACKGROUND

The present disclosure relates in general to magnetic resonance imaging (MRI) assisted methods and systems, and more particularly relates to improved methods and apparatus for performing needle biopsy of a patient's breast using MRI assisted methods.

When a substance such as human tissue is subjected to a uniform magnetic field (polarizing field B₀), the individual magnetic moments of the spins in the tissue attempt to align with this polarizing field, but precess about it in random order at their characteristic Larmor frequency. If the substance, or tissue, is subjected to a magnetic field (excitation field B₁) which is in the x-y plane and which is near the Larmor frequency, the net aligned moment, M_{z}, may be rotated, or “tipped”, into the x-y plane to produce a net transverse magnetic moment M. A signal is emitted by the excited spins after the excitation signal B₁ is terminated, this signal may be received and processed to form an image.

When utilizing these signals to produce images, magnetic field gradients (Gₓ, G_{y} and G_{z}) are employed. Typically, the region to be imaged is scanned by a sequence of measurement cycles, or “views”, in which these gradients vary according to the particular localization method being used. The resulting set of received MRI signals are digitized and processed to reconstruct the image using one of many well-known reconstruction techniques.

Intra-operative MR imaging is employed during a medical procedure to assist the doctor in guiding an instrument. For example, during a medical procedure the MRI system is operated in a real-time mode in which image frames are produced at a high rate so that the doctor can monitor the location of the needle during insertion and throughout the procedure. A locator device such as that described in U.S. Pat. Nos. 5,622,170 and 5,617,857 may be used to track the location of the instrument and provide coordinate values to the MRI system which enable it to mark the location of the instrument in each reconstructed image. The position of the medical instrument is detected by surrounding sensors. For example, the handpiece may emit light from two or more light emitting diodes which is sensed by three stationary cameras.

Tracking devices which employ the MRI system to locate markers in the medical device have also been developed. As described in U.S. Pat. Nos. 5,271,400; 5,307,808; 5,318,025; 5,353,795 and 5,715,822, such tracking systems employ a small coil attached to a catheter or other medical device to be tracked. An MR pulse sequence is performed using the tracking coil to acquire a signal which indicates the location of the tracked device. The location of the tracking coil is determined and is superimposed at the corresponding location in a medical image acquired with the same MRI system.

To accurately locate the tracking coil, position information is obtained in three orthogonal directions that require at least three separate measurement acquisitions. To correct for errors arising from resonance offset conditions, such as transmitter maladjustment and susceptibility effects, two measurements may be made in each direction with the polarity of the readout gradient reversed in one measurement. This tracking method requires that six separate measurement pulse sequences be performed to acquire the tracking coil location. As disclosed in U.S. Pat. No. 5,353,795, these separate measurements can be reduced to four in number by altering the readout gradients in a Hadamard magnetic resonance tracking sequence.

One of the primary interventional medical procedures which employ MR imaging is MRI-guided breast biopsies. Typically, these procedures are conducted without real-time MRI imaging guidance and are lengthy (45-60 minutes) complicated procedures, in part due to physical space limitation within cylindrical magnet MRI systems and the need of positioning of the breast at magnet isocenter for imaging. In the majority of these types of systems, a patient is first imaged in the MRI scanner, and images are reviewed to determine lesions/problem areas. For the biopsy, the subject breast is compressed, with a plate on one side of the breast and a coarse, MRI compatible grid on the other, and the breast/grid combination is imaged. The grid is visible in the images, and may be seen relative to the lesion, thus providing a reference to lesion position. Then, with the patient at the home position (i.e. the MRI table completely outside the bore of the magnet), and with the breast still enclosed in the grid, a biopsy is performed manually with the grid providing guidance for the biopsy device. The grid is of relatively coarse resolution, and also does not provide guidance on the angulation or depth of the needle being used in the biopsy. Due to the lack of precise 3D localization of the lesion, it is necessary that a large sample be extracted from the patient, likely more than would be required if the biopsy needle was well localized relative to the lesion. Biopsy procedures that utilize the above-described methods can also be a lengthy procedure, with the patient in and out of the magnet several times, to insure the needle is positioned right next to the lesion.

Accordingly, an improved system is needed to perform MRI assisted breast biopsies. More particularly, an improved system for performing MRI assisted breast biopsies is needed that provides a faster, more accurate, less invasive procedure in an attempt to provide greater patient comfort at a reduced cost.

### BRIEF DESCRIPTION

In accordance with one exemplary embodiment of the present disclosure a breast biopsy system is disclosed. The biopsy system includes a needle biopsy device, a MRI compatible device localization system and a Magnetic Resonance Imaging (MRI) system. The needle biopsy device includes an operating end including a biopsy needle having a needle tip. The needle biopsy device is configured for guidance by a robotic guidance device into a treatment position wherein the needle tip is positioned adjacent target tissue in patient. The MRI compatible device localization system is adapted to track one or more points on the needle biopsy device and generate real-time device localization data. The MRI system is adapted for applying a static magnetic field having substantially uniform amplitude over a target tissue in a patient and acquiring multi-planar reference image data from the patient being treated. The MRI system is connected to the MRI compatible device localization system and operable to display an overlay image reconstructed from the real-time device localization data on the multi-planar reference image data which depicts the location of the needle biopsy device relative to the target tissue in the patient.

In accordance with another exemplary embodiment of the present disclosure a breast biopsy system is disclosed. The biopsy system includes a needle biopsy device including an operating end including a biopsy needle with a needle tip. The needle biopsy device is configured for guidance by an operator into a treatment position wherein the needle tip is positioned adjacent target tissues in patient. The needle biopsy device further includes a tracking coil mounted proximate the needle tip and operable to acquire tracking data and a robotic guidance device adapted for guiding the needle biopsy device into the treatment position. The breast biopsy system further includes a Magnetic Resonance Imaging (MRI) system for acquiring multi-planar reference image data from the patient being treated. The MRI system is connected to the tracking coil for acquiring tracking data from the tracking coil as the needle biopsy device is guided into the treatment position by the robotic guidance device. The MRI system is operable to display an overlay image reconstructed from the acquired tracking data on the multi-planar reference image data which depicts the location of the needle biopsy device in the patient.

In accordance with another exemplary embodiment of the present disclosure a method of performing a robotically assisted MRI breast biopsy is disclosed. The method includes preparing a patient for the intervention by positioning the patient at a home position relative to a Magnetic Resonance Imaging (MRI) system and a robotic guidance device adapted for guiding a needle biopsy device into target tissue relative to the patient. Utilizing algorithms an optimal needle approach for the needle biopsy device and placement of the robotic guidance device relative to a treatment position are determined. The robotic guidance device is next positioned at an approximate position relative to the target tissue and the patient is advanced to a scan position in the MRI system. Multi-planar reference images of the patient are acquired to identify a lesion position on the reference images. Next, an MRI compatible device localization system is enabled to provide real-time device localization of the needle biopsy device. A real-time representation of the needle biopsy device is next displayed as an overlay on the multi-planar reference images. The robotic guidance device is next guided, based on the real-time representation of the needle biopsy device, to advance the needle biopsy device toward the targeted lesion for biopsy.

### DRAWINGS

These and other features and aspects of embodiments of the present invention will become better understood when the following detailed description is read with reference to the accompanying drawings in which like characters represent like parts throughout the drawings, wherein:

FIG. 1 is a perspective view of one embodiment of the breast biopsy system of the present disclosure, according to one or more embodiments shown or described herein;

FIG. 2 is a schematic diagram of preferred embodiment of an needle biopsy device, according to one or more embodiments shown or described herein; and

FIG. 3 is a flow chart of the preferred method of performing a breast biopsy which employs the breast biopsy system, according to one or more embodiments shown or described herein.

### DETAILED DESCRIPTION

The present disclosure is directed to a system and method for performing a breast biopsy utilizing MRI imaging systems that employ guidance and tracking means of a biopsy needle device. In particular, embodiments of the present disclosure provide a breast biopsy system including a needle biopsy device configured for guidance by a robotic guidance device into a treatment position wherein a needle tip is positioned adjacent to the target tissues in a patient. A MRI compatible device localization system is provided to track one or more points on the needle biopsy device and generate real-time device localization data. During operation, the Magnetic Resonance Imaging (MRI) system acquires multi-planar reference image data from the patient being treated. An overlay image is reconstructed from the generated real-time device localization data onto the multi-planar reference image data, thereby depicting the location of the needle biopsy device relative to the target tissue in the patient.

One or more specific embodiments of the present disclosure will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present invention, the articles “a,” “an,” “the,” and “said” are intended to mean that there are one or more of the elements. The terms “comprising,” “including,” and “having” are intended to be inclusive and mean that there may be additional elements other than the listed elements.

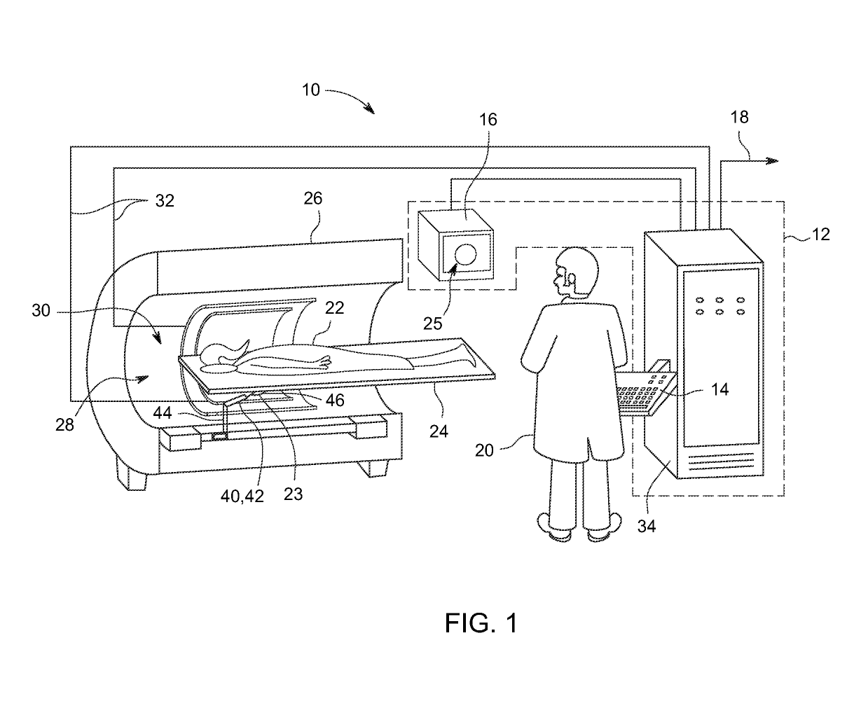

Referring first to FIG. 1, there is shown the major components of a preferred breast biopsy system 10 which incorporates the present disclosure. The operation of the system is controlled from an operator console 12 which includes an operator interface 14, such as a keyboard, joystick and/or control panel, and a display 16. The console 12 communicates through a link 18 with a separate computer system, not shown, that enables an operator 20 to control the production and display of images on the display 16. In an embodiment, the computer system may include a number of modules which communicate with each other through a backplane. These include an image processor module, a CPU module and a memory module, known in the art as a frame buffer for storing image data arrays. The computer system may be linked to a disk storage and a tape drive for storage of image data and programs, and it communicates with a separate system control through a high speed serial link. Further description of such example computer systems and included modules that may be used to control the production and display of images on the display 16 may be found in U.S. Pat. No. 6,289,233, entitled “High Speed Tracking of Interventional Devices Using an MRI System,” which is assigned to the same assignee and incorporated by reference herein.

As illustrated in FIG. 1, a patient 22 on a support table 24 is placed in a standard magnet system 26 including a bore 28, having an imaging device 30, including imaging electronics 32 coupled to an imaging and tracking unit 34. In an embodiment, the standard magnet system 26 is a Magnetic Resonance Imaging (MRI) system adapted for applying a static magnetic field having substantially uniform amplitude over a target tissue 23 in a patient 22. The system 10 is configured to acquire multi-planar reference image data 25 from the patient 22 being treated. An invasive device 40, shown in FIG. 1 as a needle biopsy device 42, is guided for insertion into the patient 22 by a robotic guidance device (described presently). In alternate embodiments, the invasive device 40 may be a catheter, a guide wire, an endoscope, a laparoscope, or similar device.

Referring still to FIG. 1, the present disclosure includes the invasive device 40, and more particularly the needle biopsy device 42, that is guided into the target tissue 23 of the patient 22, while positioned in the bore 28 of the magnet system 26, so that a biopsy of the target tissue 23 may be performed. While a conventional MRI system may be used to implement the procedure and device disclosed herein, in the preferred embodiment an MRI system that is designed to allow access by an operator guided robotic guidance device 44 is employed. When an intra-operative MR imaging procedure is conducted, the patient 22 is placed in the magnet system 26 and a region of interest, such as a breast 46 of the patient 22 is aligned near a system isocenter. The operator 20 standing proximate the magnet system 26 has unrestricted access to the region of interest in the patient via the robotic guidance device 44 and the operator console 12. The robotic guidance device 44 is a MRI compatible robot capable of operating within the limited bore space of the imaging magnet system 26. The robotic guidance device 44 is constructed to be capable of operation within the high magnetic field of the imaging magnet, and also to not generate image artifacts while the scanner is imaging. The robotic guidance device 44 is computer controlled for semi-automatic operation, or may be manually manipulated by the operator 20 using a joystick or other appropriate controls, such as operator interface 14.

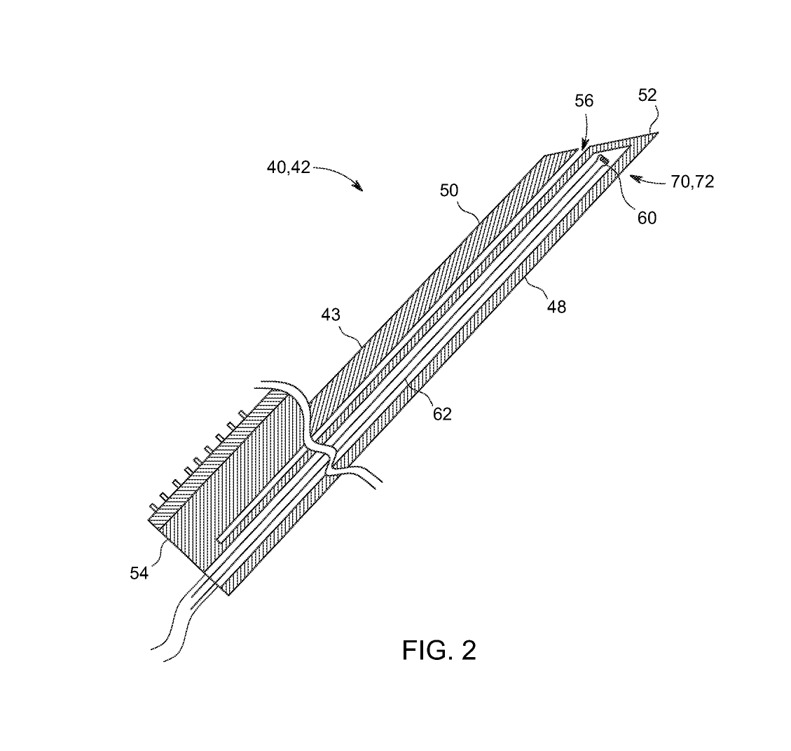

Referring now to FIG. 2, in an embodiment the invasive device 40, and more particularly the needle biopsy device 42, preferably comprises an operating end 43, including a modified soft tissue thin wall biopsy needle 48, and, for example, can be a stainless steel needle having a length of 5.5 inches (or any length greater than the depth of the lesion) from tip to base and diameter of 0.62 mm consistent with a 22 gauge Westcott biopsy needle, (or any diameter sufficient to provide for operation as disclosed herein). The needle biopsy device 42 has a shaft 50 with a tip 52 at one end and a base 54 at the other end. Preferably tip 52 is cut at an angle consistent with commercially available biopsy needles for easy insertion into the target tissue 23. A bore 56 is formed in the biopsy needle 48 for collection of a tissue sample. The biopsy needle 48 is designed for insertion either by itself or through an ultra-thin wall (such as a 20 gauge) introducer needle (not shown) into a test region, shown as tissue of the breast 46 of the patient 22 (FIG. 1).

The breast biopsy system 10 of FIG. 1 further includes an MRI compatible device localization system (described presently). To provide such localization system, the invasive device 40 of FIG. 2, and in this particular embodiment, the needle biopsy device 42, further includes a coil 60 encased in the shaft 50 of the biopsy needle 48. The coil 60 detects MR signals generated in the patient 22 responsive to the radiofrequency field created by an external coil of the magnet system 26. Since the RF coil is small, the region of sensitivity is also small. Consequently, the detected signals have Larmor frequencies which arise only from the strength of the magnetic field in the immediate vicinity of the coil 60. These detected signals are sent to the imaging and tracking unit 34 where they are analyzed. The position of the invasive device 40 is determined in the imaging and tracking unit 34 and is displayed on the display 16 by superposition, or overlay, as an overlay image 27 of the invasive device 40 on a conventional MR image, and more particularly the multi-planar reference MR image data 25 taken prior to placement of the invasive device 40 within the patient 22. In an alternative embodiment, the image of the invasive device 40 is superimposed or overlayed, on diagnostic images obtained from an imaging means prior to placement of the invasive device 40 within the patient 22, which may be an x-ray, a computed tomography (CT), a Positron Emission Tomography or ultra-sound imaging device. Other embodiments of the disclosure may image the precise location of the invasive device 40 as a graphic symbol, or the like.

Referring again to FIG. 2, as previously indicated, the invasive device 40, and more specifically the needle biopsy device 42, is designed for insertion into the patient 22 and includes the small tracking coil 60 mounted proximate to the tip 52. The tracking coil 60 has a plurality of turns, and typically may have from 1 to 20 turns. It may be as small as 1 mm in diameter. The invasive device 40 may, for example, in an embodiment be part of a catheter such as that described in U.S. Pat. Nos. 5,271,400 and 5,353,795 or an RF catheter such as that described in U.S. Pat. No. 5,437,277. The tracking coil 60 is small and it has a small region of sensitivity that picks up MRI signals from excited spins only in its immediate vicinity. The needle biopsy device 42 further comprises one or more conductors 62 mounted in the needle biopsy device 42 and coupled to the tracking coil 60 and the MRI system 26. The conductors 62 extend from the operating end 42 toward a non-operating end 45 of the needle biopsy device. The acquired MRI signals are conveyed by the pair of conductors 62 to the imaging and tracking unit 34 in the MRI magnet system 26 where they are analyzed.

As disclosed, the MRI compatible device localization system 70 is capable of providing precise and accurate real-time device localization data 72. There are multiple technologies available for device localization, but the disclosed localization system 70 is required to track multiple points on the invasive device 40, such that device orientation can be established. More particularly, in an embodiment, a method of use includes interleaving the tracking coil measurement acquisitions with the acquisition of image data. MRI tracking data is then acquired and Fourier transformed by an array processor. The transformed MRI tracking data is used by the imaging and tracking unit 34 as the real-time device localization data 72 to produce an icon representing the invasive device 40 for display on the display 16. The icon is overlaid on the MRI image of the patient anatomy at the location indicated by the tracking coil 60. As described in U.S. Pat. No. 5,353,795 issued on Oct. 11, 1994 and entitled “Tracking System To Monitor The Position Of A Device Using Multiplexed Magnetic Resonance Detection”, which is incorporated herein by reference, errors arising from resonance offset conditions make it necessary to acquire more than three tracking coil measurements.

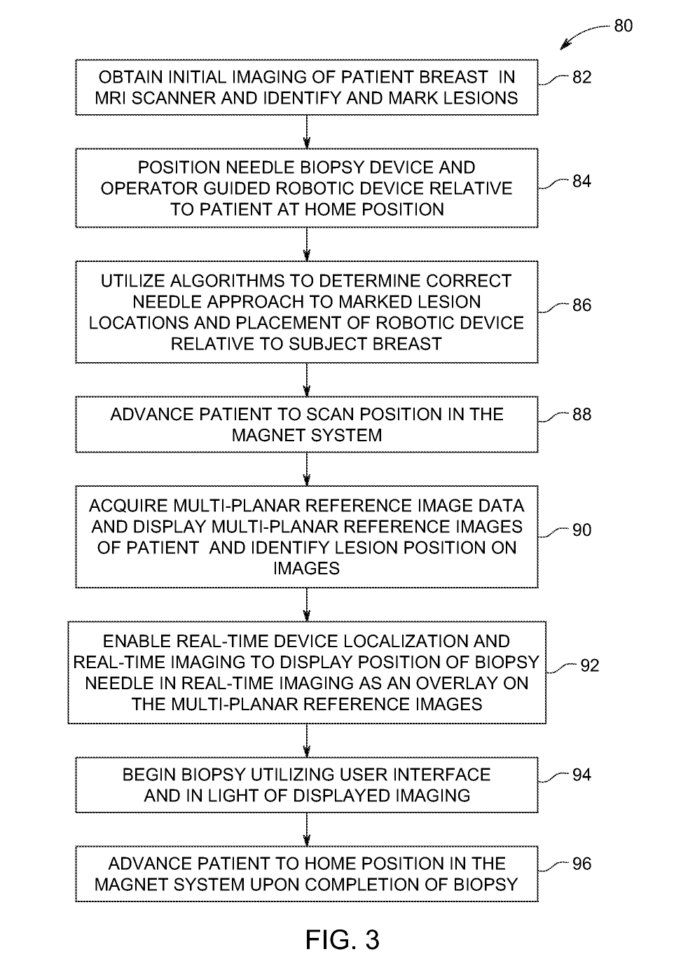

A breast biopsy utilizing the breast biopsy system according to the preferred embodiment of the disclosure is carried out by a series of steps depicted in FIG. 3. During this biopsy procedure, the breast biopsy system 10 initially acquires image data and reconstructs images of the patient which are produced on the display 16. The breast biopsy system 10 also periodically acquires tracking signals from the tracking coil 60 in the invasive device 40 being guided by the robotic guidance device 44, calculates the position of the tracking coil 60 and overlays an image or icon of the invasive device 40 on the image being displayed in display 16. The operator 20 uses this display and robotic guidance device 44 to guide the invasive device 40 into the desired position in the patient 22 with its tip 52 in contact with the tissue to be biopsied. Alternatively, the system 10 utilizing algorithms may automatically generate guidance signals for automated guidance of the robotic guidance device 44 and insertion of the biopsy needle device 42 relative to the target tissue 23.

Referring particularly to FIG. 3, indicated are the steps in the method of a biopsy procedure 80 utilizing that breast biopsy system 10 as disclosed herein. Initially, prior to the biopsy procedure, possibly days before, the patient breast 46 is imaged with a contrast agent in the MRI scanner, at step 82. In an alternative step, imaging may take place without the use of a contrast agent. The MRI images are reviewed by a radiologist, or the like, for potential lesions and lesions/targeted tissue are marked. After a determination that a biopsy is required, the patient 22 and magnet system 26 are prepared for the intervention by initial positioning of the needle biopsy device 42 and the robotic guidance device 44, while the patient 22 and patient cradle are at the home position (patient outside of magnet), in a step 84. Next, in a step 86, algorithms are utilized to determine the optimal needle approach to the marked lesion/target tissue locations, and to provide suggested positioning of the robotic guidance device 44 relative to the subject breast 46 while considering the constraints of the magnet system 10. The operator 20 next, in step 88, positions the robotic guidance device 44 at an approximate position, the patient 22 is advanced to scan position in the magnet system 26, and the biopsy procedure begins. The multi-planar reference image data 25 of the patient 22 is acquired with a contrast agent, displayed as reference images and the lesion position is identified, in a step 90. At this point, the operator 20 identifies lesions of interest on the reference images and marks them. The operator 20, in a step 92, enables the biopsy system 10, which provides real-time device localization, and a representation of the invasive device 40 is displayed as an overlay on the multi-planar reference images. Real-time imaging is also enabled, displaying images from an operator selected plane. More specifically, during this time, the operator 20 may choose from a variety of real-time imaging options, such as choosing an imaging plane with a field-of-vision (FOV) encompassing the entire breast 46, a specialized imaging plane perpendicular to the tip 52 of the biopsy needle 48, or an imaging plane that is in-plane with the biopsy needle 48. It is also possible to display the position of the biopsy needle 48 on real-time images. Finally, in a step 94, the operator 20, utilizing the operator interface 14, such as the control panel 14, begins the actual biopsy or collecting of the target tissue 23.

The disclosed breast biopsy system is a closed loop feedback system utilizing real-time device position/orientation that provides guidance of the invasive device 40 via the robotic guidance device 44, thereby providing automatic guidance of the needle biopsy device 42 to each targeted lesion. The robotic guidance device 44, in response to operator input or automated signals, based on the feedback from the real-time representation of the invasive device 40 on multi-planar images, advances the biopsy needle 48 toward the targeted lesion. Simultaneously, the operator 20 may be viewing the real-time images of the imaging plane at the tip 52 of the biopsy needle 48, allowing the needle's stopping position to be precisely positioned relative to the lesion. As the system advances the biopsy needle 48 in an automated, semi-automated or manual state of operation, the operator 20 observes the procedure, verifying the correct operation of the system 10, and retains the ability to stop the procedure or assume the control of the invasive device 40, and more particularly the needle biopsy device 42, using a joystick or other control, such as control panel 14. Subsequent to completion of the biopsy procedure, the patient 22 is advanced back to the home position in the magnet system 26, in a step 96.

Many variations are possible from the preferred embodiment described above. For example, the invasive device 40, and more particularly the biopsy needle 48 could be tracked using methods other than MR tracking in conjunction with a robotic guidance device. Also, the invasive device 40 could incorporate additional diagnostic components such as endoscopes, or the like. Alternatively, the invasive device 40 could incorporate therapeutic components such as a cryo-therapy channel or access for a cutting tool.

Accordingly, disclosed herein is breast biopsy system for robotically assisted breast biopsies and method of preforming a biopsy using the breast biopsy system. The disclosed breast biopsy system provides a high precision localization system in conjunction with an operator guided robotic guidance device enabling the ability to perform the entire biopsy procedure in situ with less tissue removal, reduced procedure time, increased patient comfort and reduced cost (reduced time from operator, such as interventional radiologist). While several presently preferred embodiments of the breast biopsy system have been described in detail herein, many modifications and variations will now become apparent to those skilled in the art. It is, therefore, to be understood that the appended claims are intended to cover all such modifications and variations as fall within the true spirit of the disclosure.

It is to be understood that not necessarily all such objects or advantages described above may be achieved in accordance with any particular embodiment. Thus, for example, those skilled in the art will recognize that the systems and techniques described herein may be embodied or carried out in a manner that achieves or improves one advantage or group of advantages as taught herein without necessarily achieving other objects or advantages as may be taught or suggested herein.

While the technology has been described in detail in connection with only a limited number of embodiments, it should be readily understood that the specification is not limited to such disclosed embodiments. Rather, the technology can be modified to incorporate any number of variations, alterations, substitutions or equivalent arrangements not heretofore described, but which are commensurate with the spirit and scope of the claims. Additionally, while various embodiments of the technology have been described, it is to be understood that aspects of the specification may include only some of the described embodiments. Accordingly, the specification is not to be seen as limited by the foregoing description, but is only limited by the scope of the appended claims.

1. A breast biopsy system comprising:
a needle biopsy device having an operating end including a biopsy needle, the biopsy needle including a needle tip, the needle biopsy device configured for guidance by a robotic guidance device into a treatment position wherein the needle tip is positioned adjacent a target tissue in a patient;
an MRI compatible device localization system adapted to track one or more points on the needle biopsy device and generate real-time device localization data;
a Magnetic Resonance Imaging (MRI) system adapted for applying a static magnetic field having substantially uniform amplitude over the target tissue in the patient and acquiring multi-planar reference image data from the patient being treated, the MRI system being connected to the MRI compatible device localization system and operable to display an overlay image reconstructed from the real-time device localization data on the multi-planar reference image data which depicts the location of the needle biopsy device relative to the target tissue in the patient.

2. The breast biopsy system as claimed in claim 1, further comprising an imaging and tracking unit configured to analyze the acquired real-time device localization data and generate the overlay image depicting the location of the tip of the needle biopsy device.

3. The breast biopsy system as claimed in claim 2, wherein the MRI compatible device localization system comprises a tracking coil mounted proximate the needle tip, said tracking coil operable to acquire the real-time device localization data as the needle biopsy device is guided into the treatment position.

4. The breast biopsy system as claimed in claim 3, wherein the needle biopsy device further comprises one or more conductors mounted in the needle biopsy device and coupled to the tracking coil and the MRI system, the conductors extending from the operating end toward a non-operating end of the needle biopsy device.

5. The breast biopsy system as claimed in claim 1, wherein the robotic guidance device is adapted to receive control signals in response to operator input.

6. The breast biopsy system as claimed in claim 1, wherein the robotic guidance device is adapted to receive control signals in response to automated data generated by the MRI system.

7. The breast biopsy system as claimed in claim 1, wherein the breast biopsy system is a closed loop feedback system utilizing real-time device position to provide guidance to the robotic guidance device.

8. The breast biopsy system as claimed in claim 1, wherein the breast biopsy system includes multiple interchangeable imaging planes.

9. The breast biopsy system as claimed in claim 8, wherein the multiple interchangeable imaging planes include an imaging plane including a field-of-view encompassing the target tissue in the patient, an imaging plane perpendicular to a tip of a needle of the needle biopsy device, and an imaging plane in-plane with a needle of the needle biopsy device.

10. A breast biopsy system comprising:
a needle biopsy device comprising:
an operating end including a biopsy needle, the biopsy needle including a needle tip, the needle biopsy device configured for guidance by an operator into a treatment position wherein the needle tip is positioned adjacent target tissues in patient;
a tracking coil mounted proximate the needle tip, said tracking coil being operable to acquire tracking data; and
a robotic guidance device adapted for guiding the needle biopsy device into the treatment position; and
a Magnetic Resonance Imaging (MRI) system for acquiring multi-planar reference image data from the patient being treated, the MRI system being connected to the tracking coil for acquiring tracking data from the tracking coil as the needle biopsy device is guided into the treatment position by the robotic guidance device, the MRI system being operable to display an overlay image reconstructed from the acquired tracking data on the multi-planar reference image data which depicts the location of the needle biopsy device in the patient.

11. The breast biopsy system as claimed in claim 10, wherein the needle biopsy device further comprises one or more conductors mounted in the needle biopsy device and coupled to the tracking coil and the MRI system, the conductors extending from the operating end toward a non-operating end of the needle biopsy device.

12. The breast biopsy system as claimed in claim 10, wherein the robotic guidance device is adapted to receive control signals in response to operator input.

13. The breast biopsy system as claimed in claim 10, wherein the robotic guidance device is adapted to receive control signals in response to automated data generated by the MRI system.

14. The breast biopsy system as claimed in claim 10, wherein the MRI system further comprises an imaging and tracking unit.

15. The breast biopsy system as claimed in claim 10, wherein said imaging and tracking unit is configured to analyze the acquired tracking data and generate the overlay image depicting the location of the tip of the needle biopsy device.

16. The breast biopsy system as claimed in claim 10, wherein the breast biopsy system is a closed loop feedback system utilizing real-time device position to provide guidance to the robotic guidance device.

17. A method of performing a robotically assisted MRI breast biopsy comprising:
preparing a patient for the intervention by positioning the patient at a home position relative to a breast biopsy system comprising a Magnetic Resonance Imaging (MRI) system and a robotic guidance device adapted for guiding a needle biopsy device into target tissue relative to the patient;
utilizing algorithms to determine an optimal needle approach for the needle biopsy device and placement of the robotic guidance device relative to a treatment position;
positioning the robotic guidance device at an approximate position and advancing the patient to a scan position in the MRI system;
acquiring multi-planar reference images of the patient to identify a lesion position on the reference images;
enabling an MRI compatible device localization system to provide real-time device localization data of the needle biopsy device;
displaying a real-time representation of the needle biopsy device as an overlay on the multi-planar reference images;
providing guidance to the robotic guidance device, based on the real-time representation of the needle biopsy device, to advance the needle biopsy device toward the targeted lesion for biopsy.

18. The method as claimed in claim 17, wherein the breast biopsy system is a closed loop feedback system utilizing real-time device position to provide guidance to the robotic guidance device.

19. The method as claimed in claim 17, wherein as the breast biopsy system advances the needle biopsy device, an operator observing the procedure maintains the ability to verify a correct operation of the system and ability to stop the procedure and assume control of the needle biopsy device using an operator interface.

20. The method as claimed in claim 17, wherein an operator observing the procedure has the ability to choose an imaging plane, wherein the imaging plane is one of a field-of-view encompassing an entire treatment area relative to the patient, a specialized imaging plane perpendicular to a tip of a needle of the needle biopsy device, or an imaging plane in-plane with a needle of the needle biopsy device.
